(19) United States
(12) Patent Application Publication
(10) Pub. No.: US 2025/0143958 A1
(43) Pub. Date: May 8, 2025

(54) VIBRATING ROLLER WITH INTEGRATED THERAPY ELEMENTS

(71) Applicant: HYPERICE IP SUBCO, LLC, Irvine, CA (US)

(72) Inventors: Alexander Joseph Aguiar, San Diego, CA (US); Robert Glen Edwards, San Diego, CA (US); Trevor Austin Kerth, San Diego, CA (US); Robert Marton, Yorba Linda, CA (US); Anthony Katz, Laguna Nigel, CA (US)

(21) Appl. No.: 18/932,123

(22) Filed: Oct. 30, 2024

Related U.S. Application Data
(60) Provisional application No. 63/595,500, filed on Nov. 2, 2023.

(51) Int. Cl. A61H 15/02; A61H 15/00; A61H 23/02; A61N 5/06

(52) U.S. Cl. CPC A61H 15/02; A61H 15/0085; A61H 23/0263; A61N 5/0613; A61H 2015/0007; A61H 2015/0014; A61H 2015/0042; A61H 2015/0071; A61H 2201/0169; A61H 2201/0207; A61H 2201/0214; A61H 2201/0228; A61H 2201/10; A61H 2201/1215; A61H 2201/5064; A61H 2201/5082; A61H 2201/5084; A61N 2005/0626; A61N 2005/0651; A61N 2005/0663

(57) ABSTRACT

Disclosed herein are therapy systems including a rolling element. The therapy systems can include a plurality of individually activatable therapy elements disposed on the rolling element. The therapy systems can include a sensor disposed on the rolling element and configured to detect a portion of the rolling element in contact with a user's body. The plurality of individually activatable therapy elements can be selectively activated based on an output of the sensor.

### CROSS-REFERENCE TO RELATED APPLICATIONS

This application claims priority and benefit from U.S. Provisional Application No. 63/595,500, titled “Vibrating Roller with Integrated Therapy Elements” and filed on Nov. 2, 2023, which is hereby incorporated by reference herein in its entirety.

### TECHNICAL FIELD

The present disclosure relates generally to the physical therapy and/or vibration therapy field, and more specifically a muscle vibrating roller with integrated therapy technology.

### BACKGROUND

Foam rolling can be used to help relieve muscle tightness, reduce lactic acid in the muscles, muscle fibrosis (adhesions and scar tissue), and risk of injury, and increase the flexibility of the muscles and tissue mobilization. Increased muscle tone and tightness can be achieved by applying pressure to the muscles via the roller.

### SUMMARY

Vibrating rollers provide benefits of increasing blood flow, increasing oxygen and nutrient consumption by muscles and improving regeneration of damaged tissues. A vibrating roller that uses a foam roller can provide cushioning and comfort to a user.

Integrating therapy elements such as vibration, heating, cooling, red light, and/or LED light therapy elements into a conventional foam roller can require significant power usage and can be inefficient if they are provided around the entire 360 degree circumference of the roller at the same time. For examples, powering elements that are not contacting a portion of a user's body consumes unnecessary energy in a large portion of the roller. An alternative design with better efficiency is to selectively activate the therapies or treatment (e.g., heat treatment) in a limited section of the roller actually contacting the user's body. For devices in which different segments are contacting the user's body at different times, continuous treatment requires activation and de-activation of various segments depending on the segments contacting the user's body at a given time.

Disclosed herein are devices, systems, and methods that relate to a muscle vibrating roller with integrated therapy technology. In general, the devices, systems, and methods as described herein include a vibrating roller with integrated therapy technology such as vibration, heating, cooling, red light, LED light, and/or other treatment technologies. For example, the vibrating roller can provide one or more treatments by activating individual therapy elements positioned at one or more segmented sections of the roller that are in contact with a user's body while deactivating therapy elements at other segmented sections of the roller. As a result, power can be efficiently provided to the device which, among other benefits, can prolong the battery life (or in some cases enable usable of a smaller battery, e.g., having smaller form factor). In some embodiments, the improvements to the therapeutic roller are disclosed herein with respect to exemplary embodiments of a system and a method.

Disclosed herein is a therapy system, comprising: a rolling element; a plurality of individually activatable therapy elements disposed on the rolling element; and a sensor disposed on the rolling element and configured to detect a portion of the rolling element in contact with a user's body, wherein the plurality of individually activatable therapy elements are selectively activated based on an output of the sensor.

In some embodiments, the rolling element includes a vibrating roller made of silicone.

In some embodiments, the rolling element includes a shape selected from the group consisting of: a cylinder, a sphere, a hemisphere, a cube, a cuboid, a cone, a torus, an ellipsoid, or a polyhedron.

In some embodiments, the rolling element includes a plurality of segmented sections, wherein at least one of the plurality of individually activatable therapy elements is positioned on each segmented section.

In some embodiments, two or more segmented sections are activatable at the same time.

In some embodiments, the rolling element is adapted to provide waterproofing, thermal insulation, or sound insulation.

In some embodiments, the sensor is an accelerometer, wherein the accelerometer can detect a section that is pointing upwards from the ground.

In some embodiments, the system further comprises one or more additional sensors selected from the group consisting of: a pressure sensor, a temperature sensor, a vibration sensor, a position sensor, an orientation sensor, a humidity sensor, a force sensor, a light sensor, or combinations thereof.

In some embodiments, the temperature sensor is configured to monitor one or more activable therapy elements so as to provide a heat treatment below or equal to a threshold temperature.

In some embodiments, the system further comprises a control system comprising one or more of wires, circuits, and user interfaces.

In some embodiments, the rolling element comprises a plurality of segmented sections, wherein at least one of the plurality of individually activatable therapy elements is positioned on each segmented section, and wherein the control system is adapted to predict a segmented section that touches the user's body, and wherein the plurality of individually activatable therapy elements on the segmented section that touches the user's body are pre-activatable.

In some embodiments, the plurality of individually activatable therapy elements comprise one or more vibration elements, heating elements, cooling elements, red light elements, LED elements, or combinations thereof.

In some embodiments, the heating elements comprises coils.

In some embodiments, the control system receive the output of the sensor to activate at least one of the plurality of individually activatable therapy elements.

In some embodiments, the control system is configured to modulate one or more treatment parameters comprising temperature, mode, and treatment time.

In some embodiments, each segmented section comprises an individual control system so as to provide a selected level of therapy in each segmented section.

In some embodiments, at least one segmented section comprises one of transparent materials, translucent materials and opaque materials.

In some embodiments, at least one segmented section comprises one or more cavities, wherein the plurality of individually activatable therapy elements comprise one or more red light elements, LED elements or combinations thereof positioned in the cavities.

In some embodiments, the system further comprises a heat spreader to transfer heat from the system to a user's body.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates a front perspective view of a vibrating roller, in accordance with some embodiments, as described herein;

FIG. 2 illustrates a rear perspective view of the vibrating roller, in accordance with some embodiments, as described herein;

FIG. 3 illustrates a front perspective view of the cylindrical roller structure with the vibration mechanism removed from the longitudinal central core, in accordance with some embodiments, as described herein;

FIG. 4 illustrates a front elevational view of the cylindrical roller structure of FIG. 3, in accordance with some embodiments, as described herein;

FIG. 5 illustrates a front perspective view of the vibration mechanism removed from the cylindrical roller, in accordance with some embodiments, as described herein;

FIG. 6 illustrates a rear perspective view of the vibration mechanism removed from the cylindrical roller, in accordance with some embodiments, as described herein;

FIG. 7 illustrates a right side elevational view of the vibration mechanism of FIGS. 5 and 6, in accordance with some embodiments, as described herein;

FIG. 8 illustrates the front perspective view of the vibration mechanism of FIG. 5 with the upper shell removed to show the internal components, in accordance with some embodiments, as described herein;

FIG. 9 illustrates a top plan view of the vibration mechanism of FIG. 8, in accordance with some embodiments, as described herein;

FIG. 10 illustrates a right side elevational view of the vibration mechanism of FIG. 7 with both the upper shell and the lower shell removed, in accordance with some embodiments, as described herein;

FIG. 11 illustrates a bottom plan view of the vibration mechanism of FIG. 10, in accordance with some embodiments, as described herein;

FIG. 12 illustrates a perspective view of the drive motor and the eccentric mass, in accordance with some embodiments, as described herein;

FIG. 13 illustrates an exploded perspective view of the roller bearing assembly that supports the shaft of the eccentric mass distal from the driving motor, in accordance with some embodiments, as described herein;

FIG. 14 illustrates an assembled perspective view of the roller bearing assembly of FIG. 13, in accordance with some embodiments, as described herein;

FIG. 15 illustrates a top plan view of a vibrating roller integrated with individually activatable therapy elements, in accordance with some embodiments, as described herein;

FIG. 16 illustrates a front perspective view of the vibrating roller of FIG. 15, in accordance with some embodiments, as described herein; and

FIG. 17 illustrates a block diagram of a control system of the vibrating roller, in accordance with some embodiments, as described herein.

### DETAILED DESCRIPTION

Disclosed herein are devices, systems, and methods that relate to a muscle vibrating roller with integrated therapy technology. In some embodiments, the improvements to the therapeutic roller are disclosed herein with respect to exemplary embodiments of a system and a method. In general, the devices, systems, and methods as described herein include a vibrating roller with integrated therapy technology such as vibration, heating, cooling, red light, LED light, and/or other therapy technologies. The vibrating roller can provide one or more treatments by activating individual therapy elements positioned at one or more segmented sections of the roller that is in contact with a user's body while deactivating therapy elements at other segmented sections of the roller. Accordingly and advantageously, power can be efficiently provided and battery life can be prolonged (or in some cases enables use of a smaller battery, e.g., having a smaller form factor). In some embodiments, the improvements to the therapeutic roller are disclosed herein with respect to exemplary embodiments of a system and a method.

In some embodiments, the devices, systems, and methods as disclosed herein include a rolling element including segmented sections and individually and selectively activatable therapy elements positioned at the segmented sections to provide vibration, heat, cooling, red light, LED light, and/or other types of therapies. The devices, systems, and methods as disclosed herein can further include software intelligence and/or control systems that selectively activate one or more therapy elements at selected levels.

In some embodiments, the system disclosed herein includes a vibrating roller having a hollow core. In particular embodiments, the vibrating roller includes a cylindrical structure. In some embodiments, the vibrating roller is replaced by any structures or forms, such as a sphere, a hemisphere, a cube, a cuboid, a cone, a torus, an ellipsoid, a polyhedron (e.g., a pyramid, a triangular prism, or a tetrahedron), or any other structures or forms having at least one of a curved face, an edge, a face, and/or a vertex corner.

In some embodiments, the therapy elements can be selectively activated to operate at one of a plurality of vibrating frequencies, temperatures, and/or light frequencies so that the vibrating roller provides one or more of vibration, heating or cooling, and/or light (e.g., red light, LED light) therapies to a portion of a user's body as the body is rolled over the device.

The embodiments are disclosed for illustration of the system and the method and are not limiting except as defined in the appended claims. Although the following description is directed to particular embodiments of a vibrating therapeutic roller, it should be understood that the disclosed system and method can be applied to other embodiments of therapeutic vibrating rollers.

### Therapeutic Vibrating Roller

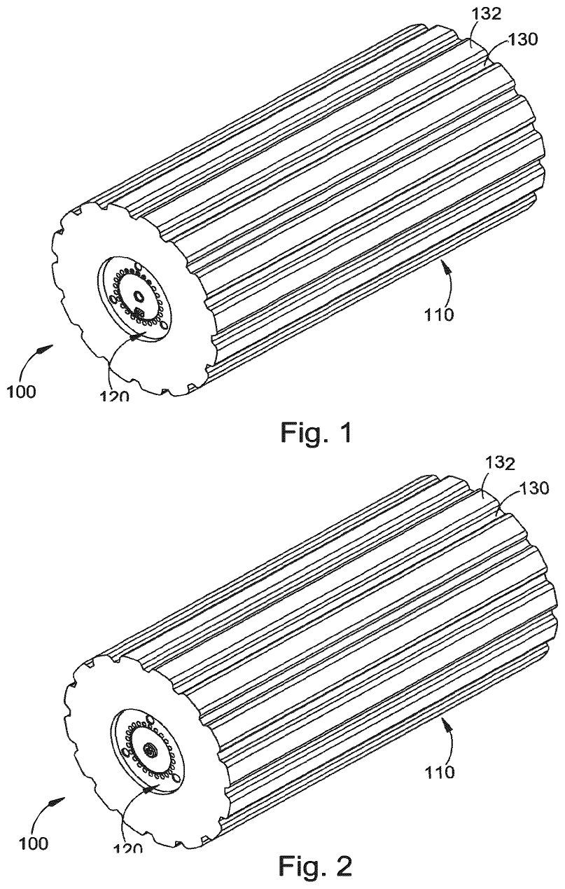

FIG. 1 and FIG. 2 illustrate a front perspective view and a rear perspective view, respectively, of a vibrating roller 100, which comprises a generally cylindrical outer roller structure 110 and an internal vibration generator 120 housed within the outer roller structure. In some embodiments, the cylindrical roller structure 110 and/or the internal vibration generator 120 can be adapted to any structures or forms as described herein.

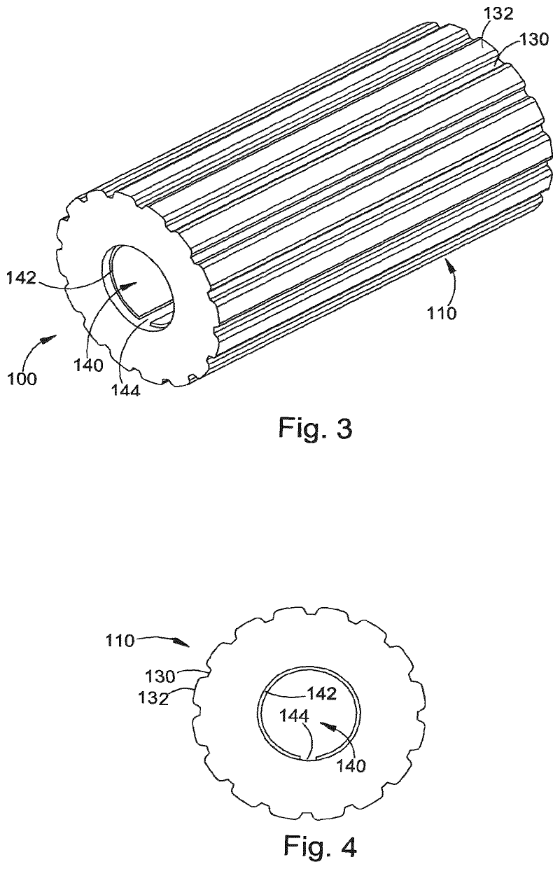

As illustrated in FIGS. 3 and 4, the outer roller structure 110 comprises a pliable foam material, such as, for example, a closed-cell polyethylene foam, or a silicone foam. For example, the foam material may comprises MINICEL® L200, L300, L380 or the like, which is commercially available from Sekisui Voltek of Lawrence, Massachusetts. The material is firm, yet is sufficiently pliable such that applying the roller to a person's body will not damage the underlying tissue.

In some embodiment, the outer roller structure 110 has an outer diameter of about 5 centimeters, about 10 centimeters, about 15 centimeters, about 20 centimeters, about 25 centimeters, about 30 centimeters, about 35 centimeters, about 40 centimeters, about 45 centimeters, or about 50 centimeters. In some embodiment, the outer roller structure 110 has a length of about 5 centimeters, about 10 centimeters, about 15 centimeters, about 20 centimeters, about 25 centimeters, about 30 centimeters, about 35 centimeters, about 40 centimeters, about 45 centimeters, or about 50 centimeters. In the illustrated embodiment, the outer roller structure 110 has an outer diameter of approximately 15 centimeters and a length of approximately 29.2 centimeters.

As further shown in FIGS. 3 and 4, the outer circumference of the outer roller structure comprises a plurality of grooves 130 that are formed to a selected depth (e.g., approximately 0.5 centimeter in the illustrated embodiment). A corresponding plurality of ribs 132 comprise the material remaining between the grooves. The smaller surface areas of the ribs allow the user to apply a greater pressure per unit area to selected portions of a body when using the roller. In the illustrated embodiment, sixteen grooves and sixteen ribs are spaced around the outer circumference of the roller structure at intervals of approximately 22.5 degrees with each rib having an angular width of approximately 16 degrees and with each groove having an angular width of approximately 6.5 degrees.

The outer roller structure 110 further includes a longitudinal central bore 140 that extends the full length of the outer shell. The diameter of the central bore is selected to receive and restrain the vibration generator 120. For example, in the illustrated embodiment, the inner diameter of the central bore and a corresponding outer diameter of the vibration generator are approximately 6 centimeters. In certain embodiments, the outer roller structure is formed by injection molding to form the grooves 130, the ribs 132 and the central bore in one step. In the illustrated embodiment, the longitudinal bore has an inner circumferential shelf 142 proximate to each end of the bore. Each shelf is recessed approximately 0.66 centimeter from the respective end of the roller structure and extends radially inward from the bore about 0.25 centimeter. A longitudinal channel 144 extends longitudinally along the inner bottom surface of the bore. The longitudinal channel has a width of approximately 1 centimeter.

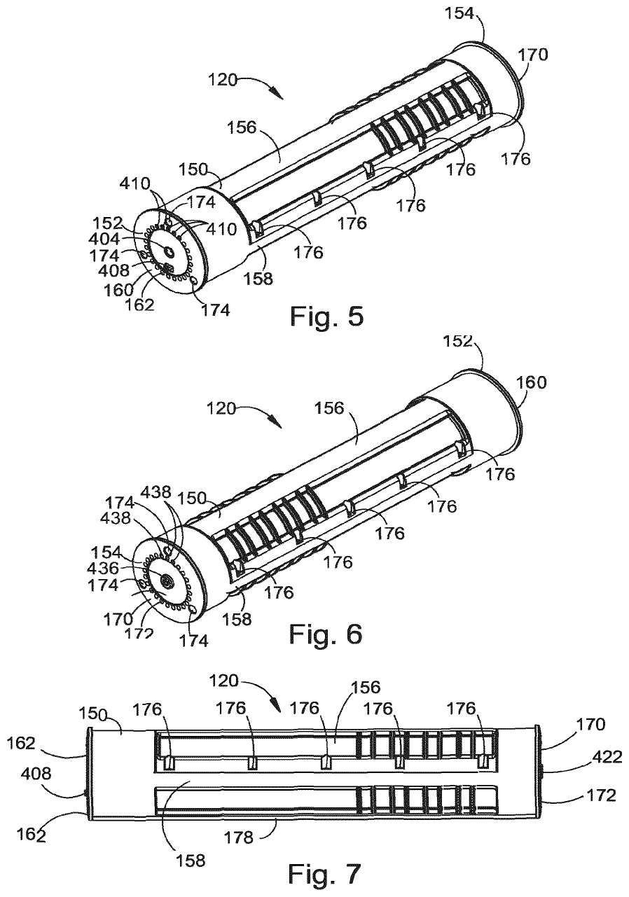

FIGS. 5, 6 and 7 illustrates a front perspective view, a rear perspective view and a right side elevational view, respectively, of the vibration mechanism 120 removed from the cylindrical roller 110. The vibration mechanism comprises a generally cylindrical outer shell 150 having a first end 152 and a second end 154. In the illustrated embodiment, the cylindrical outer shell comprises an upper shell portion 156 and a lower shell portion 158. The first end is closed by a first end cap 160, which is penetrated by a plurality of through bores 162, which provide ventilation through the first end cap. The second end is closed by a second end cap 170, which is penetrated by a plurality of ventilation through bores 172. The end caps are secured to the upper and lower shell portions by a respective plurality of screws 174. The upper shell portion is secured to the lower shell portion by a plurality of screws. 176.

The cylindrical outer shell 150 has a length of approximately 28.3 centimeters between the two end caps 160, 162 so that the cylindrical shell, which is slightly shorter than the central bore 140 of the roller structure 110. Accordingly, when installed in the roller structure, the vibration mechanism 120 does not extend beyond the ends of the roller structure, as shown in FIG. 1. The foam material of the roller structure causes the inner circumference of the central bore to provide sufficient friction against the outer circumference of the vibration mechanism to restrain the vibration system within the central bore during ordinary use, while allowing the vibration system from the central bore if required for maintenance. Furthermore, the first end cap and the second end cap are screwed onto the first and second ends of the cylindrical shell after inserting the cylindrical shell into the cylindrical roller so that the two end caps are blocked from inward movement by the circumferential shelves 142 of the longitudinal bore 140 of the cylindrical roller. As shown in FIG. 7, the lower shell portion 158 has a longitudinal ridge 178 along the bottom that is positioned and sized to engage the longitudinal channel 144 of the central bore so that the cylindrical outer shell does not rotate within the central bore.

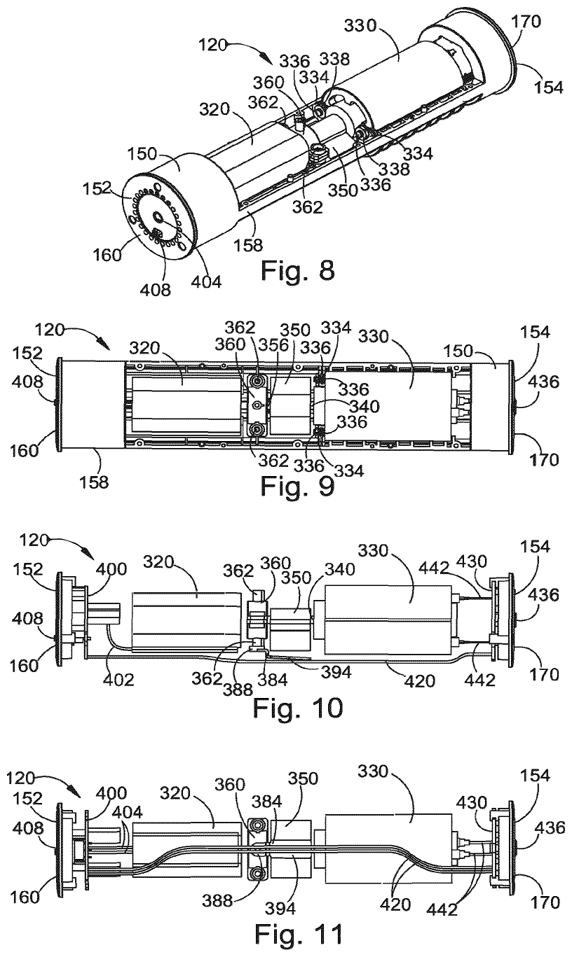

FIG. 8 illustrates the front perspective view of the vibration mechanism 120 of FIG. 5 with the upper shell 156 removed to show the internal components positioned in the lower shell 158. FIG. 9 illustrates a top plan view of the vibration mechanism of FIG. 8. FIG. 10 illustrates a right side elevational view of the vibration mechanism of FIG. 7 with both the upper shell and the lower shell removed. FIG. 11 illustrates a bottom plan view of the vibration mechanism of FIG. 10 with both the upper shell and the lower shell removed.

As shown in FIGS. 8-11, the internal components include a plurality (e.g., 4) of battery cells 320 which are electrically interconnected in series to provide a single DC output voltage. In the illustrated embodiment, the output voltage is nominally approximately 14.8 volts. In some embodiments, the cells are arranged in any desirable configuration. In some embodiments, the cells and/or the enclosure has a dimension of about 10 millimeters, about 20 millimeters, about 30 millimeters, about 40 millimeters, about 50 millimeters, about 60 millimeters, about 70 millimeters, about 80 millimeters, about 90 millimeters, about 100 millimeters, or about 200 millimeters. In some embodiments, the cells and/or the enclosure has a mass of about 10 grams, about 50 grams, about 100 grams, about 200 grams, about 300 grams, about 400 grams, about 500 grams, about 600 grams, about 700 grams, about 800 grams, about 900 grams, or about 1000 grams. In particular embodiments, the four cells are arranged in a generally rectangular, box-like enclosure (with rounded edges) having overall dimensions of approximately 70 millimeters by 42 millimeters by 38 millimeters and having a mass of approximately 200 grams. The battery cells are positioned near the first end 152 of the vibration mechanism 120. In one embodiment, the battery is a Model C1865CC-4S1P Lithium-Ion Battery commercially available from Shenkhen Bak Battery Co., Ltd. of Shenzhen, China.

A drive motor 330 is positioned near the second end 154 of the vibration mechanism. In the illustrated embodiment, the drive motor is a DC2925D012 12-volt DC electric motor commercially available from Donchang Motor (Shenzhen) Ltd. of Shenzhen, China. The drive motor has a loaded current of approximately 2.2 amperes and has a maximum loaded speed of approximately 3,250 rpm. By positioning the drive motor at the opposite end of the vibration mechanism from the battery 320, the masses of the components tend to at least partially offset so that the center of gravity of the vibration mechanism is near the center of the vibration mechanism between the two relatively massive components.

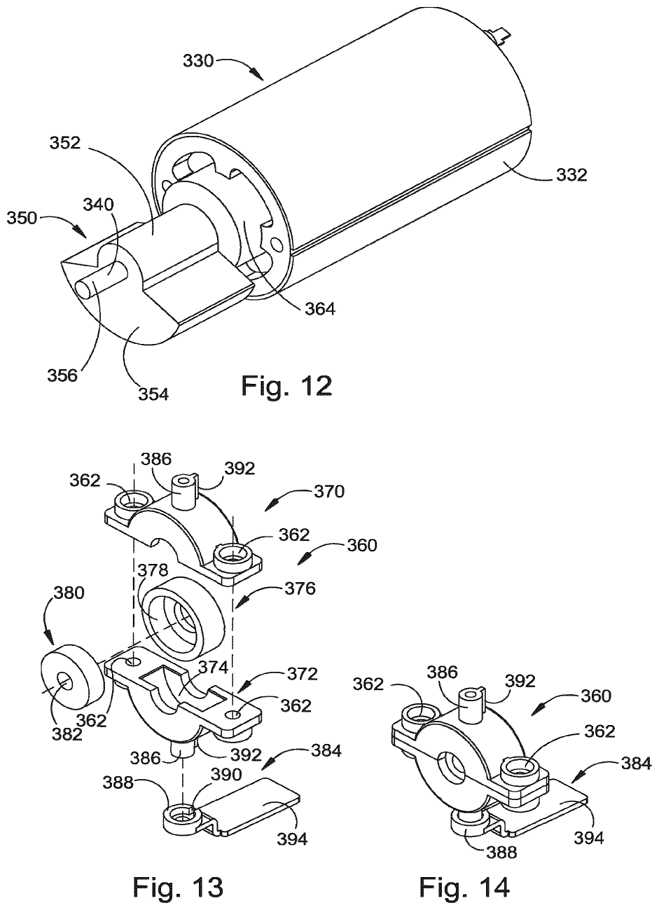

As shown in FIG. 12, the cylindrical outer perimeter of the drive motor 330 is surrounded by a generally cylindrical shockproof pad 332 to at least partially isolate the motor from physical shocks that may occur when the vibration mechanism is dropped or moved abruptly.

As shown in FIGS. 9 and 10, the drive motor 330 is secured to a pair of vertical brackets 334 that are formed in the lower shell portion 158. The drive motor is secured by a pair of screws 336 that pass through the length of the motor and engage respective threaded nuts 338 on the opposite side of the bracket from the drive motor.

The drive motor 330 has an output shaft 340 that extends toward the center of the vibration mechanism 120. An eccentric mass 350 (shown in more detail in FIG. 12) is secured to the output shaft of the drive motor. In some embodiments, the eccentric mass is a solid of any shape having an outer radius of about 1 centimeter, about 2 centimeters, about 3 centimeters, about 4 centimeters, about 5 centimeters, or about 10 centimeters with respect to the centerline of the output shaft of the motor. In the illustrated embodiment, the eccentric mass comprises an arcuate-shaped solid having an outer radius of approximately 2.1 centimeters with respect to the centerline of the output shaft of the motor. The eccentric mass has a central cylindrical portion 352 that surrounds and engages the output shaft of the drive motor. In some embodiments, the central cylindrical portion has a radius of about 0.1 centimeter, about 0.25 centimeter, about 0.5 centimeter, about 0.75 centimeter, about 1 centimeter, about 2 centimeters, about 3 centimeters, about 4 centimeters, about 5 centimeters, or about 10 centimeters. In the illustrated embodiment, the central cylindrical portion has a radius of approximately 0.75 centimeter. A fan-shaped portion 354 of the eccentric mass extending from the central cylindrical portion to the outer radius of the eccentric mass spans an angular section of approximately 140 degrees. In some embodiments, the eccentric mass has a longitudinal length along the output shaft of the drive motor of approximately about 0.5 centimeter, about 1 centimeter, about 1.5 centimeters, about 2 centimeters, about 3 centimeters, about 3.5 centimeters, about 4 centimeters, about 4.5 centimeters, about 5 centimeters, or about 10 centimeters. In the illustrated embodiment, the eccentric mass has a longitudinal length along the output shaft of the drive motor of approximately 2.5 centimeters. In some embodiments, the eccentric mass has a mass of about 10 grams, about 50 grams, about 100 grams, about 200 grams, about 300 grams, about 400 grams, about 500 grams, about 600 grams, about 700 grams, about 800 grams, about 900 grams, or about 1000 grams. In particular embodiments, the eccentric mass includes stainless steel and has a mass of approximately 170 grams.

In some embodiments, the drive motor 330 includes one motor configured to provide vibration throughout the entire 360 degrees of the vibrating roller. In some embodiments, the drive motor 330 includes two or more individual motors positioned around the outer circumference of the drive motor 330, where each motor have an angular width to provide vibration to a segmented section of the vibrating roller with an angular width of about 15 degrees, about 30 degrees, about 45 degrees, about 60 degrees, about 90 degrees, about 120 degrees, about 150 degrees, about 180 degrees, about 210 degrees, about 240 degrees, about 270 degrees, about 300 degrees, about 330 degrees, or about 360 degrees (i.e., around the entire circumstance of the vibrating roller).

As shown in FIG. 12, an extended portion 356 of the output shaft 340 of the drive motor 330 extends approximately 1.25 centimeters beyond the distal end of the eccentric mass 350. The extended portion is supported by a roller bearing assembly 360 (shown in more detail in FIG. 13), which is secured to the lower shell portion 158 by a pair of screws (not shown) that are inserted into a pair of alignment bores 362. The lengths of the output shaft and the position of the roller bearing mechanism are selected so that the eccentric mass is positioned substantially midway between the first end 152 and the second end 154 of cylindrical outer shell 150. As illustrated in the top view of FIG. 9, the length of the output shaft of the motor from a motor bearing 364, through the eccentric mass and through the roller bearing assembly is only a few millimeters longer than the longitudinal length of the eccentric mass. Thus, the output shaft is effectively prevented from wobbling in response to the rotation of the eccentric mass, which reduces wear on the motor bearing, the motor rotor and the bearings within the roller bearing assembly.

The roller bearing assembly 360 is shown in more detail in the exploded view of FIG. 13 and the assembled view of FIG. 14. The roller bearing assembly includes an upper bearing cover 370 and a lower bearing cover 372, which are substantially identical. Each bearing cover includes the pair of alignment bores 362. Each bearing cover includes a respective semicircular cavity 374. Each cavity is sized and shaped to receive an outer roller bearing race 376. The outer roller bearing race includes a circular cavity 378 that is sized and shaped to receive an inner roller bearing 380, which has an axial bore 382 sized to receive the extended portion 356 of the output shaft 340 of the drive motor 330. The roller bearing assembly is assembled by inserting the inner roller bearing into the outer roller bearing race, and then inserting the lower portion of the outer bearing race into the semicircular cavity of the lower bearing cover. The upper bearing cover is then aligned with the lower bearing cover and closed over the upper portion of the outer bearing race. Before inserting the roller bearing assembly into the lower shell half 158, as shown in FIGS. 8 and 9, a wire protection bracket 384 is positioned onto an extended cylindrical protrusion 386 on the bottom of the lower bearing cover. The wire protection bracket includes a circular collar portion 388 that is sized to fit the extended cylindrical protrusion. The collar has a slot 390 that engages a rib 392 on the cylindrical protrusion. The collar is secured to the cylindrical protrusion by a screw (not shown). The wire protection bracket further includes a generally L-shaped plate 394 that extends from the collar such that when the roller bearing assembly is secured to the lower shell half as shown in FIGS. 8 and 9, the plate is positioned between the eccentric mass 350 as shown in FIGS. 10 and 11. The wire protection plate protects wiring from the rotating eccentric mass as described below. The identical upper bearing cover also has the cylindrical protrusion and rib; however, the protrusion and rib are not used in the illustrated embodiment.

When power is applied to the drive motor 330 to rotate the eccentric mass 350, the rotation causes extensive vibrations of the eccentric mass, which are communicated to the lower shell portion 158. The upper shell portion 156 is secured to the lower shell portion by the plurality of screws 176 (FIGS. 5-7) so that the vibrations are further communicated to the upper shell portion. Accordingly, the entire cylindrical outer shell 150 is caused to vibrate by the rotation of the eccentric mass by the drive motor. Because of the central location of the eccentric mass, the amplitudes of the vibrations are greater near the center of the cylindrical outer shell. Thus, when the cylindrical outer shell is positioned in the longitudinal central bore 140 of the outer roller structure 110 as shown in FIGS. 1 and 2, the vibrations are communicated through the outer roller structure and are concentrated on the portions of the ribs 132 nearer the longitudinal center of the outer roller structure. Thus, when providing therapeutic massage to a body part, the outer roller structure can be gripped near each end where the vibrations have lower amplitudes. The central portion of the outer roller structure, where the vibrations have greater amplitudes, is applied to the body part (e.g., an arm, leg, back, neck or shoulder muscle) needing therapy.

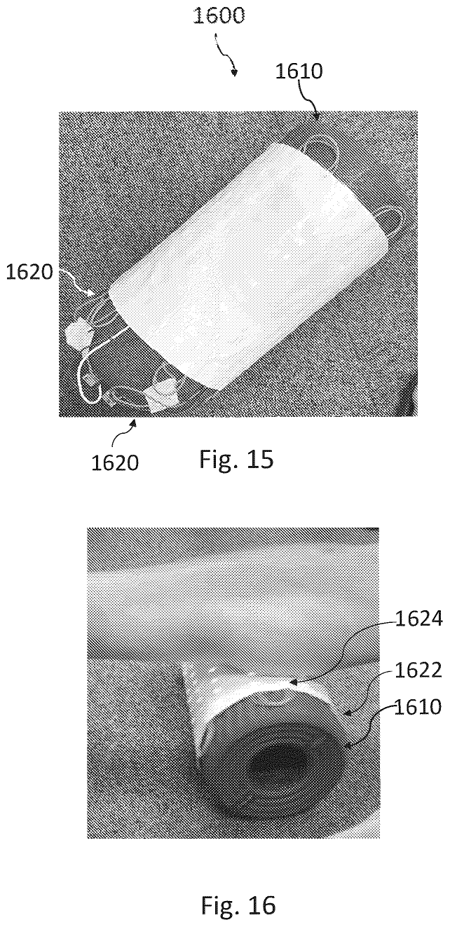

The battery 320 is electrically connected to a first circuit board 400 via a pair of wires 402. The first circuit board is secured to the first end 152 of the cylindrical outer shell 150. As shown in FIG. 8, a charging terminal 404 extends from the first circuit board and through the first end cap 160 so that the charging terminal is accessible when the cylindrical outer shell is inserted in the outer roller structure 110 (FIG. 2). The charging terminal is electrically connectable to a conventional battery charger adapter (not shown) to charge the battery when needed. The charging terminal is electrically connected to a battery charging circuit 406 (shown schematically in FIG. 15, described below). The first end cap further includes a power switch 408 that is coupled to the first circuit board. The power switch selectively electrically connects and disconnects the battery from the other circuitry (described below) to provide switched battery power to the other circuitry. In certain embodiments, the first circuit board may include a plurality (e.g., 5) LEDs 410 that extend through selected ventilation holes 162 in the first end cap (FIG. 5) to provide an indication of the charge status in the battery.

As discussed above, when operating the vibrating roller 100, a user selects an operating speed/vibration frequency for particular activities or particular parts of the body (e.g., arms, legs, neck, back or the like).

Therapeutic Vibrating Roller with Integrated Therapy Technology

In various embodiments, it is desirable to provide an additional therapy to a user, along with or separate from the vibration therapy. As described above, integrating additional therapy elements such as heating, cooling, red light, and/or LED light elements into a conventional foam roller can result in inefficient and unnecessary use of power when activating the entire 360 degrees of the roller, especially when only a portion of the roller is in contact with the user's body at a given time.

Advantageously, the configuration of a segmenting rolling element integrated with a plurality of individually and selectively activatable therapy elements can result in less power, prolonged battery life, more available selections provided to a user, and continuous and efficient treatment as compared with a conventional foam roller with one single therapy element.

FIG. 15 illustrates a top plan view of a vibrating roller integrated with individually activatable therapy elements, in accordance with some embodiments, as described herein. FIG. 16 illustrates a front perspective view of the roller of FIG. 15, in accordance with some embodiments, as described herein.

In general, the vibrating roller 1600 includes a rolling element 1610, a plurality of individually activatable therapy elements 1620, and/or a sensor (not shown) configured to detect a section of the rolling element 1610 that contacts a user's body. In some embodiments, the vibrating roller 1600 further includes one or more heat spreaders, at least one of which is an external layer of the vibrating roller 1600. In some embodiments, the vibrating roller 1600 has a weight of about 50 grams, about 100 grams, about 150 grams, about 200 grams, about 500 grams, or about 1000 grams. In some embodiments, the vibrating roller 1600 includes a dimension (e.g., diameter, length, width, height, or thickness) of about 5 centimeters, about 10 centimeters, about 15 centimeters, about 20 centimeters, about 25 centimeters, about 30 centimeters, about 40 centimeters, or about 50 centimeters.

In general, the rolling element 1610 is a roller, such as the vibrating roller 100 as described above. Accordingly, the rolling element 1610 includes the outer roller structure 110 and the internal vibration generator 120 as described above. In some embodiments, the rolling element 1610 includes any materials (e.g., polyethylene or silicone) that can provide cushioning and comfort to the user's body.

In some embodiments, the rolling element 1610 can take any shape, structure or form, such as a cylinder, a sphere, a hemisphere, a cube, a cuboid, a cone, a torus, an ellipsoid, a polyhedron (e.g., a pyramid, a triangular prism, or a tetrahedron), or any other structures or forms having at least one of a curved face, an edge, a face, and/or a vertex corner.

In some embodiments, the rolling element 1610 is configured to provide vibration around the entire 360 degrees of the vibrating roller 1610. In some embodiments, the rolling element 1610 is segmented into 2, 3, 4, 5, or more individual sections around the outer circumferences of the rolling element where each section includes an individual motor such that the vibration can be applied in a particular section of the vibrating roller 1610 while other sections of the rolling element 1610 stay silent. In some embodiments, the segmented individual sections of the rolling element 1610 include an angular width of about 15 degrees, about 30 degrees, about 45 degrees, about 60 degrees, about 90 degrees, about 120 degrees, about 150 degrees, about 180 degrees, about 210 degrees, about 240 degrees, about 270 degrees, about 300 degrees, about 330 degrees, or about 360 degrees (i.e., around the entire circumstance of the vibrating roller). In various embodiments, segments can be overlapping or non-overlapping.

In some embodiments, the rolling element 1610 is adapted to provide waterproofing, thermal insulation, and/or sound insulation.

In general, the activatable therapy elements 1620 are positioned at the outer circumference of the rolling element 1610. In some embodiments, one or a portion of the activatable therapy elements 1620 is positioned in one segmented section of the rolling element 1610 so as to provide therapy to the segmented section when the element is activated.

In some embodiments, the rolling element 1610 is segmented into 2, 3, 4, 5, or more individual sections around the outer circumferences of the rolling element 1610 where each section includes one or more individually activatable therapy elements 1620. In some embodiments, a segmented section and/or an activatable therapy element include an angular width of about 15 degrees, about 30 degrees, about 45 degrees, about 60 degrees, about 90 degrees, about 120 degrees, about 150 degrees, about 180 degrees, about 210 degrees, about 240 degrees, about 270 degrees, about 300 degrees, about 330 degrees, or about 360 degrees (i.e., around the entire circumstance of the vibrating roller). For example, the rolling element 1610 may be segmented into 2 sections, where each section includes an angular width of about 180 degrees, and where each section includes an activable element such as a coil that can individually provide heating in advance of or when the section is in contact with a user's body. In another example, the rolling element 1610 may be segmented into 4 sections, where each section includes an angular width of about 90 degrees, and where each section includes an activable element such as a coil that can individually provide heating in advance of or when the section is in contact with a user's body. In another example, the rolling element 1610 may be segmented into 6 sections, where each section includes an angular width of about 60 degrees, and where each section includes an activable element such as a coil that can individually provide heating in advance of or when the section is in contact with a user's body.

In some embodiments, at least a portion of one or more sections of the rolling element 1610, such as the outer portion of the section that the activatable therapy elements 1620 are positioned on and can heat up, comprises thermally conductive materials, such as silicone, aluminum, or any thermally conductive materials.

In some embodiments, at least a portion of one or more sections of the rolling element 1610 includes transparent or translucent materials such that light (e.g., light generated by the activatable therapy elements 1620) can pass through and provide light therapy to a user's body, alone or in combination with other types of therapies as described herein. In some embodiments, at least a portion of one or more sections of the rolling element 1610 include non-transparent or opaque materials (e.g., aluminum). In some embodiments, at least a portion of one or more sections of the rolling element 1610 includes one or more holes (or cavities) where red light elements and/or LED elements can be positioned. Accordingly, the light generated by the red light elements and/or LED elements can pass through the holes (or cavities) and provide light therapy to a user's body, alone or in combination with other types of therapies as described herein. In some embodiments, the holes (or cavities) include the same or slightly larger (e.g., 10% larger) size of one or more red light elements and/or LED elements positioned in the holes.

In some embodiments, the activatable therapy elements 1620 include one or more vibration elements (e.g., drive motor 330), heating elements (e.g., electric coils 1622, thermal alloy, wires of nichrome (also known as NiCr or nickel-chromium), carbon fiber wires, or other heat sources), cooling elements, red light elements (e.g., red light devices 1624), LED elements (e.g., LED bulbs), or combinations thereof. For example, an activatable therapy element may include an electric coil 1622 that can provide heat therapy to the user's body by converting the electrical energy to thermal energy. In another example, an activatable therapy element may include a cooling element such as a cooler that can provide cold therapy to the user's body. In another example, an activatable therapy element includes one or more light bulbs that can provide non-invasive light therapy to improve or heal skin, muscle tissues, and/or other parts of the user's body.

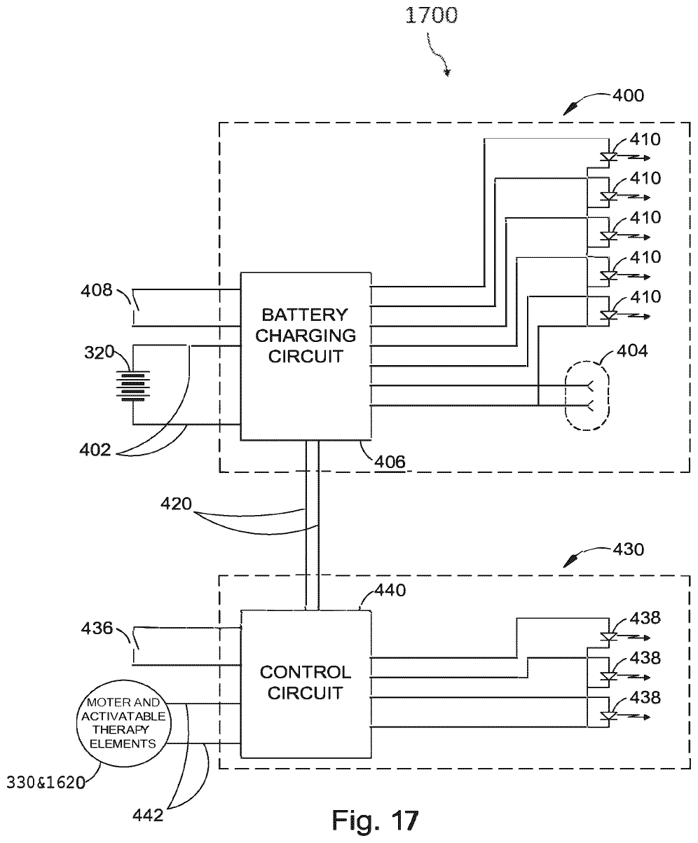

In general, an activatable therapy element can be activated by a control system 1700 (FIG. 17) based on an output signal from the sensor. In some embodiments, only the activatable therapy elements positioned on one or a subset/portion of the segmented sections of the rolling element 1610 is activated at one time. In some embodiments, the activatable therapy elements positioned on two or more sections of the rolling element 1610 are activated at the same time. For example, it may be desired to activate multiple (e.g., 2, 3, 4, or more) sections (e.g., to heat up) together at the same time so as to cover more surface area of a user's body as compared with activating only one section.

In general, a sensor is operatively (e.g., electrically) coupled to other elements (e.g., rolling element 1610 and/or activatable therapy elements 1620) and is configured to detect a section of the rolling element that is touching a user's body. In some cases, a proxy for such a portion can be the portion pointing longitudinally upwards as that is the portion contacting the user's body in a conventional use of a roller.

The sensor can be a position sensor, an orientation sensor, or any other suitable types of sensors that can detect the section of the rolling element that is in contact with the user's body. In particular embodiments, the sensor includes an accelerometer.

In some embodiments, The rolling element 1610 includes one sensor. In some embodiments, The rolling element 1610 includes two or more sensors. In some embodiments, each section of the rolling element 1610 includes one or more individual sensors.

In some embodiments, a sensor is positioned in proximity of the outer surface of the rolling element 1610, or any other suitable area of the vibrating roller 1600. In particular embodiments, the sensor is positioned axially with the activatable therapy elements 1620 such that a position that is detected by the sensor is not substantially offset from the position of an activatable therapy element that is to be activated by the output of the sensor. Therefore, when the vibrating roller starts to roll or rotate, the sensor detects the segmented section that touches the user's body (e.g., the section that is pointing upwards from the ground), and sends a signal to a control system to selectively activate one or more therapy element(s) on that section to provide therapy to the user's body. As the device continues to roll or rotate, the therapy elements at each individual section may be alternatively turned on and off such that only the therapy elements at a section that contacts the user or points upwards at the moment is activated while other sections are deactivated. In some embodiments, the types of therapy elements to be activated when in use are pre-determined or selected e.g., by the user.

In some embodiments, each segmented section of the vibrating roller includes one or more additional sensors of any type such as a pressure sensor, a temperature sensor, a vibration sensor, a position sensor, a humidity sensor, a force sensor, or a light sensor such that a selected (e.g., user-selected, or pre-determined) level of therapy can be achieved. For example, one or each section may include a temperature sensor integrated with a heating element (e.g., element 1622) at respective section. The temperature sensor at a section can monitor the temperature of the corresponding section such that it is lower than or equal to a threshold temperature. Accordingly, a selected level or heat treatment can be achieved, and adverse effects (e.g., burning of the user's body) can be avoided. In some embodiments, the temperature sensor at each section can provide the same or at least two different levels of heat. Thus, as the vibrating roller rolls with different segmented sections touching the user's body, the roller can provide the same or at least two (e.g., 2, 3, 4, 5, or more) different levels of heat around the 360 degrees of the roller to the user.

In some embodiments, an individual red light or LED element may include a plurality of light bulbs that form an array that comprises a plurality of rows and columns and positioned along the longitudinal axis of the vibrating roller with an angular width of about 15 degrees, about 30 degrees, about 45 degrees, about 60 degrees, about 90 degrees, about 120 degrees, about 150 degrees, about 180 degrees, about 210 degrees, about 240 degrees, about 270 degrees, about 300 degrees, about 330 degrees, or about 360 degrees (i.e., around the entire circumstance of the vibrating roller).

In some embodiments, a section to be in contact with a user's body can be predicted or pre-determined (e.g., by the control system of the vibrating roller). Accordingly, the activatable therapy elements in the section can be activated in advance. For example, a segmented section that is in contact with the user's body can be predicted and a heating element on that section can be pre-heated.

In general, a heat spreader of the vibrating roller 1600 can transfer energy as heat (e.g. heat generated by one or more individually activatable therapy elements 1620) from the vibrating roller 1600 to a user's body. Additional details for heat spreaders are described in U.S. patent application Ser. No. 17/308,012, which is hereby incorporated by reference in its entirety.

### Control System

The devices, systems, and methods as described herein may further include a control system or a control module that is configured to control the operation of elements of the vibrating roller (e.g., the vibration of the roller and/or the activation of therapy elements) and the interaction between the vibrating roller and a user.

In general, the control system (e.g., system 1700 in FIG. 17) includes various electric components (e.g., wires, circuits, and/or user interfaces) to communicate among elements of a vibrating roller and between the vibrating roller and a user. For example, the control system 1700 can modulate one or more treatment parameters, such as temperature (e.g., between 10° C. and 40° C.; or at about 10° C., about 15° C., about 20° C., about 25° C., about 30° C., about 35° C., and/or about 40° C.), mode (e.g., cooling modes, heating modes, red light modes, LED modes, or other suitable modes), and treatment time (e.g., between about 30 seconds and about 24 hours; or about 30 seconds, about 1 minute, about 30 minutes, about 1 hour, about 3 hours, about 6 hours, about 12 hours, or about 24 hours), and/or other treatment parameters. In some embodiments, the treatment parameters may be selected or pre-determined by the user e.g., using a remote device (e.g., a remote control, a smartphone, a smartwatch, or other suitable devices) that is in wired or wireless communication with the control system. For example, the user can select the temperature modes by using a client application on a mobile device in communication with the control system (e.g., via Bluetooth).

In some embodiments, the control system 1700 is configured to predict or determine a section to touch a user's body (e.g., as the roller rotates), and to activate the activatable therapy elements (e.g., pre-heat the section) in advance. Given commonly understood heat transfer principles, in some instances, a segment that was previously activated to a fully heated (or other therapy delivering) condition may retain some of that heat (or other energy) in a subsequent activation session. For example, a segment of the roller that transitions between contacting the user's body and not contacting the user's body during a particular exercise regime may retain energy from a prior activation session at the onset of a subsequent activation session. In some embodiments, the control system can account for this condition and deliver less power for subsequent activation sessions, as less power is needed to bring the segment back up to a fully energized state. The control system can operate in a closed loop system, e.g., using feedback from a sensor (e.g., a temperature sensor) or in an open loop system (e.g., using artificial intelligence and other learning techniques).

FIG. 17 illustrates a block diagram of a control system 1700 of the vibrating roller, in accordance with some embodiments, as described herein.

As shown in FIG. 17, the switched battery power from the first circuit board 400 is provided by a pair of wires 420 to a second circuit board 430, which is secured to the second end cap 170. When the cylindrical outer shell is assembled, the wires extending between the first circuit board and the second circuit board are positioned beneath the L-shaped plate 394 of the wire-protection bracket 384, and are thus shielded from contacting the rotating eccentric mass as shown in FIGS. 10 and 11. In some embodiments, the circuit boards (e.g., circuit boards 400 and/or 430) as described herein includes a printed circuit board (PCB). In some embodiments, the circuit boards (e.g., circuit boards 400 and/or 430) as described herein includes a flexible printed circuit board.

The second circuit board 430 is electrically connected to a power/frequency selection pushbutton switch 436, which is centered in the second end cap 170 (FIG. 6). The second circuit board is further electrically connected to one or more indicator light emitting diodes (LEDs) 438 (e.g., three), which are positioned in one or more of the plurality of ventilation through bores 172 in the second end cap (FIG. 6). The second circuit board includes a control circuit 440 (shown schematically in FIG. 15), which is responsive to the pushbutton switch to control at least 1) the rotational speed of the drive motor 330; and/or 2) the temperature, frequency of the light, or other features of the activatable therapy elements 1620 by varying the voltage provided to the drive motor 330 and/or activatable therapy elements 1620 via a pair of wires 442. The control circuit 440 thus controls 1) the frequency of the vibrations caused by the rotating eccentric mass 350, and/or 2) the level of the heating or cooling, and/or the power of light caused by the activated therapy elements 1620. Further, the control circuit 440 also selectively illuminates the indicator LEDs on the second circuit board to provide a display indicative of the selected rotational speed of the motor, selected level of heating or cooling, selected level of light, and/or any other features (e.g., time of operation, time of pre-heating, instant temperature of the activated therapy element for heating or cooling).

Further, the control circuit 440 can control the time of activation of the drive motor 330 and/or the activatable therapy elements 1620 e.g., in each individual section of the vibrating roller by varying the voltage provided to the section. In some embodiments, each section includes an individual control circuit (e.g., control circuit 440) and/or sensors so that each section can operate to provide vibration, heating, cooling, red light, LED light, or other therapies with at a selected level individually.

As shown in FIG. 17, a block diagram of the electrical connections of the first circuit board 400 and the second circuit board 430 is illustrated. As illustrated, the first circuit board includes the battery charging circuit 406 that is electrically connected to the battery 320, to the charging terminal 404 and to the first set of indicator LEDs 410. The battery charging circuit 406 receives DC power via the charging terminal and selectively provides charging current to the battery cells 320 when an active DC adapter (not shown) is connected to the charging terminal. The battery charging circuit 406 operates in a conventional manner to control the charging current to assure that the battery is not overcharged. The battery charging circuit also monitors the status of the battery and provides an indication of the charge status of the battery via the first set of indicator LEDs.

The battery charging circuit 406 is electrically connected to the control circuit on the second circuit board 430 via the wires 420 to provide DC voltage to the control circuit when the vibration circuit is selectively activated via the pushbutton power switch 408. The control circuit is responsive to the applied DC voltage to provide power to the drive motor 330 and/or the activable therapy elements 1620 via the wires 442.

As described above, the control circuit is configured to control the rotational speed of the drive motor, which in turn controls the frequency of the vibration caused by the rotating eccentric mass 350 and/or 2) the level of the heating or cooling, and/or the power of light caused by activating the activatable therapy elements 1620. In some embodiments, the control circuit is a pulse-width modulation (PWM) control circuit that provides controls by varying the duty cycles of pulses to control the power provided to the motor and/or the activatable therapy elements 1620. In some embodiments, the control circuit is responsive to repeated activations of the pushbutton switch to cycle between an off position and two or more levels of rotational speeds, temperatures, and/or light. For example, in one embodiment, the pushbutton switch selects between off and 2, 3, 4, 5, or more rotational speeds. In another example, the pushbutton switch selects between off and 2, 3, 4, 5, or more heating or cooling temperatures. In another example, the pushbutton switch selects between off and 2, 3, 4, 5, or more powers or frequencies or light.

The control circuit is electrically connected to the one or more LEDs 438 to display the selected operation. For example, in one embodiment, a single tricolor LED may be operable to selectively display red, green or blue, with each color representing an operating speed/vibration frequency. Alternatively, the single tricolor LED can be replaced with separate LEDs that represent each operating speed/vibration frequency. For example, in the embodiment illustrated in FIG. 15, three LEDs are provided to identify up to three operating speeds and corresponding vibration frequencies.

Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. One or more memories can store media assets (e.g., audio, video, graphics, interface elements, and/or other media files), configuration files, and/or instructions that, when executed by a processor, form the modules, engines, and other components described herein and perform the functionality associated with the components. The processor and the memory can be supplemented by, or incorporated in special purpose logic circuitry.

It should also be noted that the present implementations can be provided as one or more computer-readable programs embodied on or in one or more articles of manufacture. The article of manufacture can be any suitable hardware apparatus, such as, for example, a floppy disk, a hard disk, a CD-ROM, a CD-RW, a CD-R, a DVD-ROM, a DVD-RW, a DVD-R, a flash memory card, a PROM, a RAM, a ROM, or a magnetic tape. In general, the computer-readable programs can be implemented in any programming language. The software programs can be further translated into machine language or virtual machine instructions and stored in a program file in that form. The program file can then be stored on or in one or more of the articles of manufacture.

As various changes could be made in the above constructions without departing from the scope of the invention, it is intended that all the matter contained in the above description or shown in the accompanying drawings shall be interpreted as illustrative and not in a limiting sense.

All references, issued patents and patent applications cited within the body of the specification are hereby incorporated by reference in their entirety, for all purposes.

### Definitions

Terms used in the claims and specification are defined as set forth below unless otherwise specified.

The term “about” refers to a ±10% variation from the nominal value unless otherwise indicated or inferred.

The term “substantially” means ±10%, and in some embodiments, ±5%.

The term “each” includes a portion of referents unless the context clearly dictates otherwise.

The term “treatment” or “therapy” refers to any act, hobby, task, program that relieves tension, the treatment of disease or disorders by some remedial, rehabilitating, or curative process, a curative power or quality, or psychotherapy.

The term “rolling element,” “roller structure,” or similar terms refers to an object that can roll or move along a surface by revolving on an axis, as a cylinder, a sphere, or any other structures as described herein.

The term “printed circuit board” or “PCB” refers to a medium used in electrical and/or electronic engineering to connect electronic components to one another in a controlled manner. In some embodiments, a PCB is a wire board used to mechanically support and electrically connect electronic components using conductive pathways, tracks or signal traces etched from metal sheets laminated onto a non-conductive substrate.

The term “circuit” or “electric circuit” refers to a complete circular path that electricity flows through. In some embodiments, the circuit as described herein is a closed circuit. In some embodiments, the circuit as described herein is an open circuit.

The term “heat,” “warm,” “warmth,” “heating,” “warming” or similar terms are exchangeable and refer to making a place, a user, or a thing warm.

The term “user” refers to a human or non-human, male or female.

It must be noted that, as used in the specification, the singular forms “a,” “an” and “the” include plural referents unless the context clearly dictates otherwise. The indefinite articles “a” and “an,” as used in the specification and in the claims, unless clearly indicated to the contrary, should be understood to mean “at least one.”

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that some alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A therapy system, comprising:
a rolling element;
a plurality of individually activatable therapy elements disposed on the rolling element; and
a sensor disposed on the rolling element and configured to detect a portion of the rolling element in contact with a user's body,
wherein the plurality of individually activatable therapy elements are selectively activated based on an output of the sensor.

2. The system of claim 1, wherein the rolling element includes a vibrating roller made of silicone.

3. The system of claim 1, wherein the rolling element includes a shape selected from the group consisting of: a cylinder, a sphere, a hemisphere, a cube, a cuboid, a cone, a torus, an ellipsoid, or a polyhedron.

4. The system of claim 1, wherein the rolling element includes a plurality of segmented sections, wherein at least one of the plurality of individually activatable therapy elements is positioned on each segmented section.

5. The system of claim 4, wherein two or more segmented sections are activatable at the same time.

6. The system of claim 1, wherein the rolling element is adapted to provide waterproofing, thermal insulation, or sound insulation.

7. The system of claim 1, wherein the sensor is an accelerometer, wherein the accelerometer can detect a section that is pointing upwards from the ground.

8. The system of claim 1, further comprises one or more additional sensors selected from the group consisting of: a pressure sensor, a temperature sensor, a vibration sensor, a position sensor, an orientation sensor, a humidity sensor, a force sensor, a light sensor, or combinations thereof.

9. The system of claim 8, wherein the temperature sensor is configured to monitor one or more activable therapy elements so as to provide a heat treatment below or equal to a threshold temperature.

10. The system of claim 1, further comprising a control system comprising one or more of wires, circuits, and user interfaces.

11. The system of claim 10, wherein the rolling element comprises a plurality of segmented sections, wherein at least one of the plurality of individually activatable therapy elements is positioned on each segmented section, and wherein the control system is adapted to predict a segmented section that touches the user's body, and wherein the plurality of individually activatable therapy elements on the segmented section that touches the user's body are pre-activatable.

12. The system of claim 1, wherein the plurality of individually activatable therapy elements comprise one or more vibration elements, heating elements, cooling elements, red light elements, LED elements, or combinations thereof.

13. The system of claim 12, wherein the heating elements comprises one or more coils, thermal alloy, nichrome wires, carbon fiber wire, or combinations thereof.

14. The system of claim 13, wherein the control system receive the output of the sensor to activate at least one of the plurality of individually activatable therapy elements.

15. The system of claim 13, wherein the control system is configured to modulate one or more treatment parameters comprising temperature, mode, and treatment time.

16. The system of claim 4, wherein each segmented section comprises an individual control system so as to provide a selected level of therapy in each segmented section.

17. The system of claim 4, wherein at least one segmented section comprises one of transparent materials, translucent materials and opaque materials.

18. The system of claim 4, wherein at least one segmented section comprises one or more cavities, wherein the plurality of individually activatable therapy elements comprise one or more red light elements, LED elements or combinations thereof positioned in the cavities.

19. The system of claim 1, further comprises a heat spreader to transfer heat from the system to a user's body.
